(19) United States
(12) Patent Application Publication
(10) Pub. No.: US 2024/0087707 A1
(43) Pub. Date: Mar. 14, 2024

(54) MEDICAL CARE ASSISTANCE SYSTEM, MEDICAL CARE ASSISTANCE DEVICE, MEDICAL CARE ASSISTANCE METHOD, AND RECORDING MEDIUM

(71) Applicant: OMRON HEALTHCARE CO., LTD., Kyoto (JP)

(72) Inventors: Hironori SATO, Kyoto (JP); Yoko KANEMITSU, Kyoto (JP)

(21) Appl. No.: 18/511,458

(22) Filed: Nov. 16, 2023

Related U.S. Application Data
(63) Continuation of application No. PCT/JP2022/026620, filed on Jul. 4, 2022.

(30) Foreign Application Priority Data
Jul. 9, 2021 (JP) 2021-114500

(51) Int. Cl. G16H 20/10; G16H 70/20

(52) U.S. Cl. CPC G16H 20/10; G16H 70/20

(57) ABSTRACT

A medical care assistance system includes guideline selection means that selects a guideline related to treatment of a patient based on predetermined input information, patient information acquisition means that acquires patient information including measured value information related to biological information on the patient and medication information as information of a medicine prescribed to the patient, determination means that performs determination related to appropriateness of a current treatment strategy including a prescription content of a medicine for the patient based on the selected guideline and the patient information, recommended treatment information generation means that generates recommended treatment information regarding an action recommended to be performed on the patient according to a determination result of the determination means, and output means that outputs the recommended treatment information.

### CROSS-REFERENCE TO RELATED APPLICATIONS

This application is the U.S. national stage application filed pursuant to 35 U.S.C. 365(c) and 120 as a continuation of International Patent Application No. PCT/JP2022/026620, filed Jul. 4, 2024, which application claims priority to Japanese Patent Application No. 2021-114500, filed Jul. 9, 2021, which applications are incorporated herein by reference in their entireties.

### TECHNICAL FIELD

The present invention belongs to a technical field related to healthcare, and relates to a medical care assistance system, a medical care assistance device, a medical care assistance method, and a program (recording medium).

### BACKGROUND ART

Conventionally, a device (system) as described in Patent Literature 1 is known as a technique for assisting a doctor or the like to prescribe a medicine in medical care. Patent Literature 1 proposes a medication assistance device that assists deciding a medicine to be prescribed to a patient by calculating recommendation scores of medicines to be prescribed based on a medical care guideline in consideration of a medical history and contraindication information, and presenting prescription plans (medication candidates) based on the recommendation scores for the patient at a first visit.

However, there has been a problem in such a conventional system that it cannot be applied to a patient who is already taking medication. For this reason, it is not possible to cope with a case where a new complication occurs in a patient who is taking medication, a case where treatment has already been performed by another doctor (hospital), such as a patient who has been transferred from another hospital, or the like. Also, in an opposite case, it is not possible to cope with a case where a patient's medical condition is improved by improving lifestyle habits or removing a factor, such as stress, that has been worsening a disease.

### CITATION LIST

### Patent Literature

Patent Literature 1: JP 2018-156149 A

### SUMMARY OF INVENTION

### Technical Problem

In view of the above-described problems, an object of the present invention is to provide a technique that allows changing a prescription for a patient who is already taking medication in a system that assists medical care of medical professionals.

### Solution to Problem

The present invention adopts the following configurations to solve the above-described problems. That is, a medical care assistance system includes:
guideline selection means that selects a guideline related to treatment of a patient based on predetermined input information;
patient information acquisition means that acquires patient information including measured value information related to biological information on the patient and medication information as information of a medicine prescribed to the patient;
determination means that performs determination related to appropriateness of a current treatment strategy including a prescription content of a medicine for the patient based on the selected guideline and the patient information; recommended treatment information generation means that generates recommended treatment information regarding an action recommended to be performed on the patient according to a determination result of the determination means; and
output means that outputs the recommended treatment information.

Here, the “predetermined input information” includes a medical history of the patient, an international disease classification code (ICD code) related to a disease, a biochemical test result of the patient, other various test results, and the like. Also, the “guideline” is assumed to be, for example, medical care guidelines defined by various institutes according to diseases, but are not necessarily limited to such guidelines. For example, it may be a unique guideline determined from knowledge obtained by an individual doctor. The “prescription content” is a content, such as amount, type, and combination of medicine(s), and includes a prescription pattern obtained by compiling the content. Also, the “action recommended to be performed on the patient” includes not only changing the prescription content but also actions other than medication, for example, performing various tests or changing the applied guideline. Also, the “output means” may be a display device, such as a liquid crystal display, or may be a printing device, such as a printer.

According to such a configuration, it is possible to determine whether or not the current prescription content (medication status) is appropriate based on the guideline and the current state of the patient, and upon that, present taking an appropriate (that is, recommended) action when the current prescription content is not appropriate. For this reason, it is possible to propose an appropriate action including a change of the prescription content as necessary even for a patient who is already taking medication.

Further, the determination means may determine at least whether or not the prescription content currently adopted is excessive for a medical condition of the patient based on the patient information, and the recommended treatment information generation means may generate the recommended treatment information that recommends reduction in type and/or amount of medicine prescribed to the patient when the prescription content currently adopted is excessive for the medical condition of the patient. With such a configuration, in a case where the prescription content is excessive for the current medical condition of the patient, it is possible to recommend reduction in type/amount of medicine.

Further, the determination means may determine at least whether or not the medicine according to the prescription content currently adopted is replaceable with a compounded medicine, and the recommended treatment information generation means may generate the recommended treatment information that recommends a prescription change to the compounded medicine when the medicine according to the prescription content currently adopted is replaceable with the compounded medicine. According to such a configuration, even in the case of a prescription having no problem from the viewpoint of medicine efficacy, in a case where there is a combination of medicines that is replaceable with a compounded medicine, it is possible to recommend a prescription change that can reduce a burden related to medicine ingestion of the patient by presenting the compounded medicine.

Further, the guideline may define a recommended prescription content according to a medical condition, the determination means may determine at least whether or not the prescription content currently adopted matches the recommended prescription content, and the recommended treatment information generation means may generate the recommended treatment information that recommends a prescription change to the recommended prescription content when the prescription content currently adopted does not match the recommended prescription content. According to such a configuration, when a prescription having content different from the prescription content recommended by the guideline is performed, it is possible to present the fact and information that recommends a prescription with the recommended prescription content to a doctor.

Further, the guideline may define a recommended prescription content according to a medical condition, the patient information may include a predetermined improvement target reference related to the biological information, the determination means may determine at least whether the prescription content currently adopted matches the recommended prescription content, and whether the measured value information has achieved the improvement target reference, and the recommended treatment information generation means may generate, when the measured value information has not achieved the improvement target reference and the prescription content currently adopted matches the recommended prescription content, the recommended treatment information that recommends a prescription change based on the recommended prescription content one step ahead in the guideline or the recommended treatment information that recommends a test to be performed on the patient.

Here, the “improvement target reference” may be a general-purpose normal value or the like regarding the target biological information, or may be a value decided each time according to the patient's state, a prescription, or the like.

According to the configuration described above, it is possible to recommend an appropriate action to a patient who has not achieved the improvement target reference even though medication has been performed in compliance with the recommended prescription pattern according to the guideline. That is, when a recommended prescription content (prescription pattern) is defined in a guideline, it is usual that a minimum prescription pattern is recommended for type/amount of the medicines at an initial stage of treatment, and a recommended prescription pattern in which the type and amount are increased incrementally according to a treatment effect (or a medical condition) is defined. Therefore, when the treatment target has not been achieved even though the medication is performed in compliance with the recommended prescription pattern according to the guideline, the medication according to the recommended prescription pattern one step ahead is recommended. On the other hand, there is also assumed a possibility that the guideline currently applied to the patient is not appropriate in the first place (due to occurrence of a complication or the like), and in such a case, it is necessary to change the guideline to an appropriate one. Therefore, it is recommended to perform a test as necessary based on patient data including the measured value and the prescription content, and it is proposed to consider changing the guideline.

Further, the medical care assistance system may further include non-recommended medicine determination means that determines presence or absence of a non-recommended medicine that is unrecommended to be prescribed to the patient based on the patient information, and the recommended treatment information generation means may generate, when the non-recommended medicine is present, the recommended treatment information that recommends a prescription change to a prescription content excluding the non-recommended medicine.

Here, the “non-recommended medicine” refers to a medicine corresponding to a contraindication or an unacceptable medicine (such as a medicine complained about a side effect from a patient) and a combination thereof. With such a configuration, it is possible to present a prescription change with the prescription content excluding a non-recommended medicine that should not be prescribed to the patient.

In addition, the predetermined input information may include an international disease classification code (ICD code) related to a disease of the patient. According to this, it is possible to efficiently select a guideline to be applied by a code for specifying a disease.

Further, the biological information may include at least a blood pressure value, and a disease targeted for treatment of the patient may be hypertension. Since hypertension treatment usually takes a long period and there are other diseases to which blood pressure values are related, the present invention that allows recommending a review of the guideline and the prescription pattern to be applied according to periodic test results with respect these diseases is preferable.

In addition, the present invention can also be considered as a medical care assistance device including the guideline selection means, the patient information acquisition means, the determination means, and the recommended treatment information generation means, and constituting at least a part of the medical care assistance system according to any one of claims 1 to 8.

Further, the present invention can also be considered as a program (recording medium) for causing a computer to function as such a medical care assistance device, and a computer-readable recording medium including such a program recorded therein in a non-transitory manner.

Further, the present invention can also be considered as a medical care assistance method including:
selecting a guideline related to treatment of a patient based on predetermined input information;
acquiring patient information including measured value information related to biological information of the patient and medication information as information of a medicine prescribed to the patient;
performing determination related to appropriateness of a current treatment strategy including a prescription content of a medicine for the patient based on the selected guideline and the patient information;
generating recommended treatment information regarding an action recommended to be performed on the patient according to a determination result of the performing; and
outputting the recommended treatment information.

Also, the configurations and processing described above can be combined with one another to constitute the present invention unless the combination leads to contradiction.

### Advantageous Effects of Invention

With the present invention, there can be provided a technique that allows changing a prescription for a patient who is already taking medication in a system that assists medical care of medical professionals.

### BRIEF DESCRIPTION OF DRAWINGS

FIG. 1 is a schematic diagram illustrating a configuration of a medical care assistance system 1 according to an example.

FIG. 2 is a flowchart schematically illustrating a general flow of hypertension treatment performed in the example.

FIG. 3 is a block diagram illustrating a functional configuration of a server device according to the example.

FIG. 4 is an explanatory diagram indicating one example of guidelines and prescription patterns in the example.

FIG. 5 is a block diagram illustrating a functional configuration of a doctor-side terminal according to the example.

FIG. 6 is a first diagram describing a screen example output on the doctor-side terminal.

FIG. 7 is a second diagram describing a screen example output on the doctor-side terminal.

FIG. 8 is a third diagram describing a screen example output on the doctor-side terminal.

FIG. 9 is a flowchart illustrating one example of processing performed by the medical care assistance system according to the example.

FIG. 10 is a diagram illustrating an outline of a medical care assistance system 2 according to a modified example of the example.

### DESCRIPTION OF EMBODIMENTS

### Example 1

Hereinafter, one example of embodiments of the present invention will be specifically described with reference to the drawings.

### System Configuration

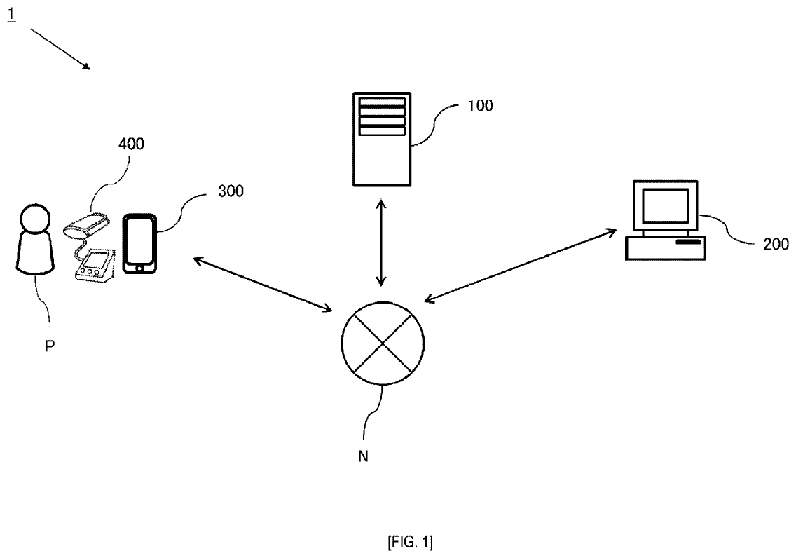

FIG. 1 is a schematic diagram illustrating a configuration of a medical care assistance system 1 according to the present example. As illustrated in FIG. 1, the medical care assistance system 1 includes a server device 100, a doctor-side terminal 200 used by a doctor, and a patient-side terminal 300 and a blood pressure monitor 400 used by a patient P. These respective configurations can communicate with one another via a communication network N.

The medical care assistance system 1 according to the present example is a system used for remote medical care. The medical care assistance system 1 transmits a blood pressure value measured by a patient at home to the server device 100 via the network N, processes various kinds of information including the measured value, and provides the processed information to a doctor, thereby assisting the doctor in performing treatment on the patient.

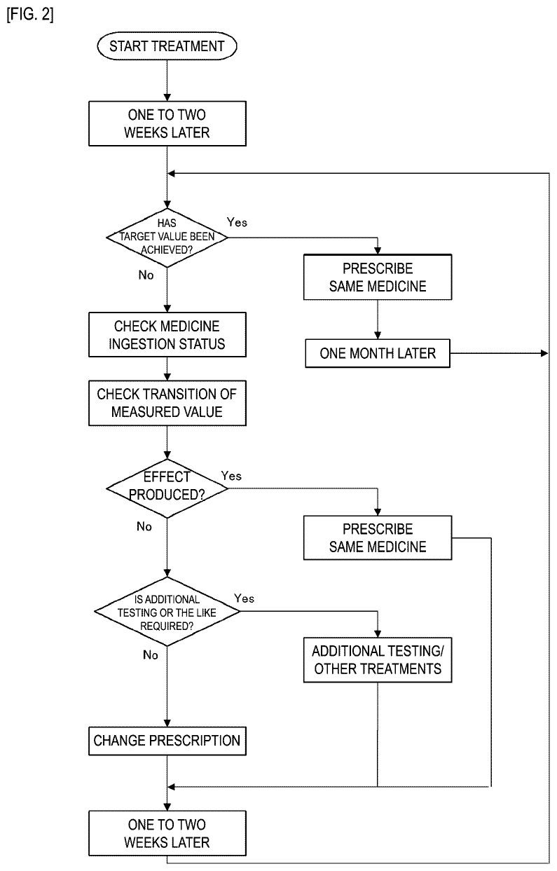

In the present example, since a treatment performed on a patient with hypertension is described as an example, a flow related to the treatment of hypertension will be described based on FIG. 2. FIG. 2 is a flow chart schematically illustrating one example of a flow of the treatment performed in the treatment of hypertension. As illustrated in FIG. 2, a patient who has received a diagnosis of hypertension starts medication treatment according to a doctor's prescription, and continuously measures his/her blood pressure at home by him/herself. Then, after a predetermined period has elapsed (for example, after one to two weeks), it is determined whether or not the measured values have achieved a target value set in advance (hereinafter, the fact that the measured value has achieved the target value is also expressed as “being controlled” or the like). Here, the measured value having achieved the target value means, for example, that the blood pressure value is less than a predetermined threshold. When the measured value is in a controlled state, the treatment is continued with the same prescription content, and after a predetermined period (for example, one month) has elapsed, it is determined whether or not the measured value of blood pressure is in a controlled state.

On the other hand, when the target value has not been achieved, the medication status is checked to see whether the patient has been taking the medicine according to the prescription. In addition, the transition of the measured value at each predetermined period is checked, and is used for deciding on a treatment strategy together with the information on the medication status.

When the doctor determines from the state of transition of the measured value that a certain effect is produced by the medication even though the target value has not been achieved, the doctor decides to continuously prescribe the same medicine. In that case, it is determined again after a predetermined period (for example, one to two weeks) has elapsed, whether or not the measured value of blood pressure is in a controlled state.

Also, when the medication does not seem to be effective, the possibility of other diseases is considered, and it is decided to perform additional tests or treatment (including prescription) taking into account the other diseases. In that case, it is determined again after a predetermined period (for example, one to two weeks) has elapsed, whether or not the measured value of blood pressure is in a controlled state.

In addition, when other diseases are not the causes and the state is expected to improve by changing the medicine being taken, such as stopping the medication due to a side effect, the prescription content is changed. In that case, it is determined again after a predetermined period (for example, one to two weeks) has elapsed, whether or not the measured value of blood pressure is in a controlled state.

By repeating the flow described above, the treatment of the hypertensive patient is executed. The medical care assistance system 1 according to the present example assists in performing such treatment and reduces the burden on the doctor (medical professional). Hereinafter, the respective configurations of the system will be described.

### Server Device

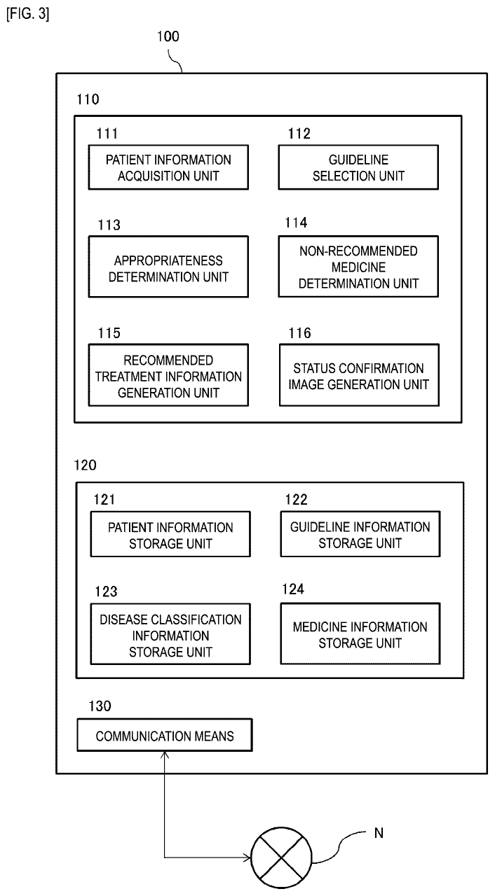

FIG. 3 is a block diagram illustrating a functional configuration of the server device 100. The server device 100 is constituted of a general server computer and includes a control unit 110, storage means 120, and communication means 130.

The control unit 110 is means that manages the control of the server device 100, and is constituted of a processor, such as a Central Processing Unit (CPU) or a Digital Signal Processor (DSP). In addition, the control unit 110 includes respective functional units: a patient information acquisition unit 111; a guideline selection unit 112; an appropriateness determination unit 113; a non-recommended medicine determination unit 114; a recommended treatment information generation unit 115; and a status confirmation image generation unit 116 as functional modules related to medical care assistance. In the present example, the server device 100 corresponds to the medical care assistance device.

The patient information acquisition unit 111 acquires information related to the disease and treatment of the patient via the communication network N and stores the information in the storage means 120. Here, the patient information includes a medical history of the patient P input by the doctor, an international disease classification code (ICD code) related to a disease, results of a biochemical test or other tests, information on a prescribed medicine, a measured value measured by the patient P with the blood pressure monitor 400, information related to a medicine ingestion status of the patient P, and the like.

The guideline selection unit 112 is a functional unit that selects an optimal guideline for treatment of a disease of a patient based on predetermined information. The treatment content of the disease of the patient is generally decided based on medical care guidelines defined according to diseases (for example, in the case of hypertension, a hypertension treatment guideline issued by the Japanese Society of Hypertension). However, since it is often the case that other diseases are complicated, it is necessary to select an appropriate guideline (and a prescription pattern) in consideration of the complication in order to perform appropriate treatment.

Therefore, the guideline selection unit 112 specifies the disease of the patient based on various kinds of information, such as medical history, biochemical test results including HbA1c, NT-proBNP, blood potassium concentration, and serum creatinine, electrocardiographic (ECG) findings, left ventricular ejection fraction (LVEF) by echocardiogram, and an arteriosclerosis index, and selects an optimal guideline (and a prescription pattern). Specifically, for example, the guideline/prescription pattern are selected by the following determination.

That is, when a complication other than hypertension is not expected, a standard prescription pattern of a “hypertension guideline” is selected. When the patient has diabetes, or the HbA1c is equal to or higher than a predetermined value, a diabetes complication prescription pattern of the “hypertension guideline” is selected. When the patient has heart failure, the NT-proBNP is equal to or higher than a predetermined value, or LVEF<40% is satisfied, a hypertension complication prescription pattern of a “heart failure guideline” is selected. When the patient has kidney failure, or eGRF calculated from blood creatinine is equal to or higher than a predetermined value, a kidney failure complication prescription pattern of the “hypertension guideline” is selected. These pieces of guideline information may be stored in the storage means 120 in advance and be read out therefrom as described below, or may be acquired from an external database via the communication network N each time.

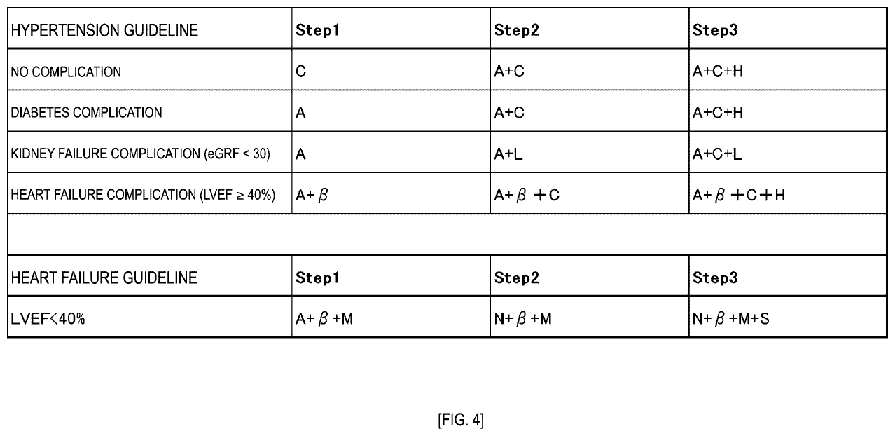

FIG. 4 indicates an example of guidelines/prescription patterns. In FIG. 4, the types of medicines are represented by symbols: A represents an ACEi/ARB; β represents a β blocker; C represents a CCB; H represents a thiazide group diuretic; L represents a loop diuretic; N represents an ARNI; M represents an MRA; and S represents a SLTG2 inhibitor.

As indicated in FIG. 4, the hypertension guideline defines, as one example, four types of prescription patterns including no complication, diabetes complication, kidney failure complication, and heart failure complication. In addition, as one example, one type of prescription pattern is defined in the heart failure guideline. In each of these prescription patterns, steps (steps 1 to 3) are set, and as the step progresses, the types of medicines included in the prescription pattern are set to increase.

The appropriateness determination unit 113 determines the appropriateness of the treatment strategy of the prescription content for a patient P based on the selected guideline/prescription pattern and patient information. Specific content of the determination will be described below. In addition, the non-recommended medicine determination unit 114 determines the presence or absence of a non-recommended medicine that is unrecommended to be prescribed to the patient P based on the patient information, medicine information to be described below, or the like. The term “non-recommended medicine” refers to a medicine corresponding to a contraindication or an unacceptable medicine (such as a medicine complained about a side effect from the patient) and a combination thereof.

Based on the determination results of the appropriateness determination unit 113 and the non-recommended medicine determination unit 114, the recommended treatment information generation unit 115 generates recommended treatment information to be suggested to a medical professional regarding an action (such as a change of a prescription content or an additional test) recommended to be performed on the patient P. The status confirmation image generation unit 116 generates a treatment status confirmation image in which measured value transition information indicating a change in the measured value measured by the patient P using the blood pressure monitor 400 at each predetermined period and information related to the medicine ingestion status of the patient are browsable. The generated recommended treatment information and treatment status confirmation image are transmitted to the doctor-side terminal 200 via the communication network N.

Although not illustrated, the storage means 120 includes a main storage unit, such as a Read only memory (ROM) or a Random access memory (RAM), and an auxiliary storage unit, such as an EPROM, a Hard Disk Drive (HDD), a Solid State Device (SSD), or a removable medium. The auxiliary storage unit stores an operating system (OS), various programs, and the like. By the stored programs being loaded into a work area of the main storage unit and executed, and the respective component units and the like being controlled by the execution of the programs, the respective functional units that meet predetermined purposes can be realized.

As illustrated in FIG. 3, the auxiliary storage unit of the storage means 120 includes a patient information storage unit 121, a guideline information storage unit 122, a disease classification information storage unit 123, and a medicine information storage unit 124. The patient information storage unit 121 stores various kinds of information acquired by the patient information acquisition unit 111. The guideline information storage unit 122 stores the above-described guideline information. The disease classification information storage unit 123 stores diseases and the international disease classification codes (ICD codes) associated with the diseases. The medicine information storage unit 124 stores information on medicines (such as the names, manufacturers, dosage forms, effects, side effects, and contraindications) and information on compounded medicines of these medicines.

The communication means 130 is communication means for connecting the server device 100 to the communication network N, and is constituted including, for example, a communication interface board and a wireless communication circuit for wireless communication.

### Doctor-Side Terminal

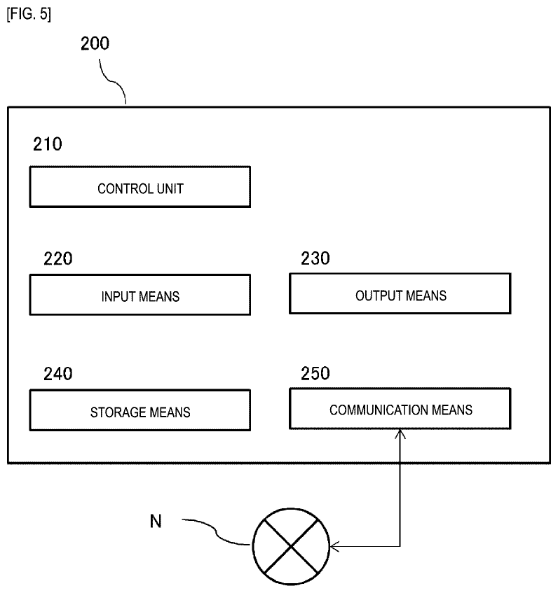

FIG. 5 is a block diagram illustrating a functional configuration of the doctor-side terminal 200. The doctor-side terminal 200 is a general computer, such as a fixed personal computer, a portable notebook-type personal computer, or a tablet device, and includes a control unit 210, input means 220, output means 230, storage means 240, and communication means 250.

The control unit 210 is means that controls the doctor-side terminal 200, and is constituted of, for example, a CPU. The input means 220 is means that receives information input from the outside, such as a keyboard, a mouse, a touch panel, a camera, or a microphone. The output means 230 is constituted including a liquid crystal display, a speaker, and the like. The storage means 240 is constituted similarly to the server device including a main storage unit, an auxiliary storage unit, and the like, and stores various data acquired via the communication network N, such as an operating system (OS) and various programs. The communication means 250 is constituted including, for example, a communication interface board and a wireless communication circuit for wireless communication.

Note that, although not illustrated, the doctor-side terminal may be accessible to an electronic medical chart management system. In such a case, electronic medical chart data of a patient stored in the electronic medical chart management system may be read out to be transmitted to the server device 100, and recommended treatment information, treatment status confirmation image and the like transmitted from the server device 100 may be associated with the electronic medical chart data. In such a case, it is also possible to allow the doctor to check the recommended treatment information and the treatment status confirmation image via the electronic medical chart management system.

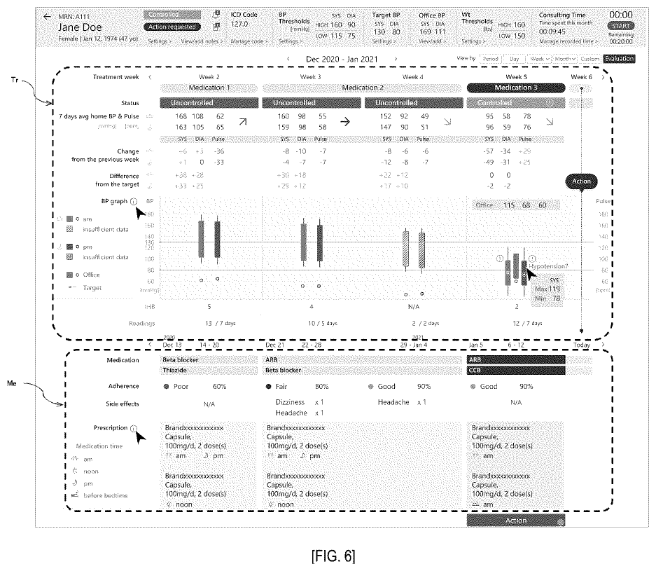

In the doctor-side terminal 200, the recommended treatment information and the treatment status confirmation image are acquired from the server device 100 via the communication network N, and these pieces of information are output to the output means 230. FIG. 6 to FIG. 8 illustrate examples of screens displayed on the output means 230 of the doctor-side terminal 200. FIG. 6 is a treatment status confirmation image of an individual patient P, and is a screen including measured value transition information Tr and medicine ingestion information Me.

As illustrated in FIG. 6, in the measured value transition information Tr of the treatment status confirmation image, the average values (7 days avg home BP & Pulse) of the systolic blood pressure (SYS), the diastolic blood pressure (DIA), and heart rate in one week are displayed with one week as a predetermined period (a period as one group). More specifically, one week average values of each SYS and DIA are displayed for each of the time periods in the morning (after getting up) and at night (before going to bed). These values are not only depicted by numerical values, but also depicted as a graph in which a range between SYS and DIA is filled in a bar shape and a relationship with a threshold line indicating a target value is visually indicated. The lines extending upward and downward from the bar-shaped portions of the graph indicate the variation of the measured values in the measurement for one week, and the line extending upward indicates the systolic blood pressure value of the week and the line extending downward indicates the diastolic blood pressure value. In addition, the week in which the bar-shaped portion is hatched indicates that the information is not reliable because the amount of the measurement data is small.

In addition, in the measured value transition information Tr, a change amount (Change from the previous week) in a case of comparing with a numerical value of the previous week and a value (Difference from the target) indicating a deviation from the target value (in this example, SYS is 130 mmHg and DIA is 80 mmHg) are also indicated.

In addition, in the medicine ingestion information Me, information such as the types of medicines ingested by the patient P in that week, the frequencies of medicine ingestion (in this example, indicated as Adherence by a dose rate), and symptoms and frequencies of complained side effects are described. In addition, information (Prescription) indicating a prescription content, such as a brand, a dosage form, and a dose amount of a medicine, can also be referred to.

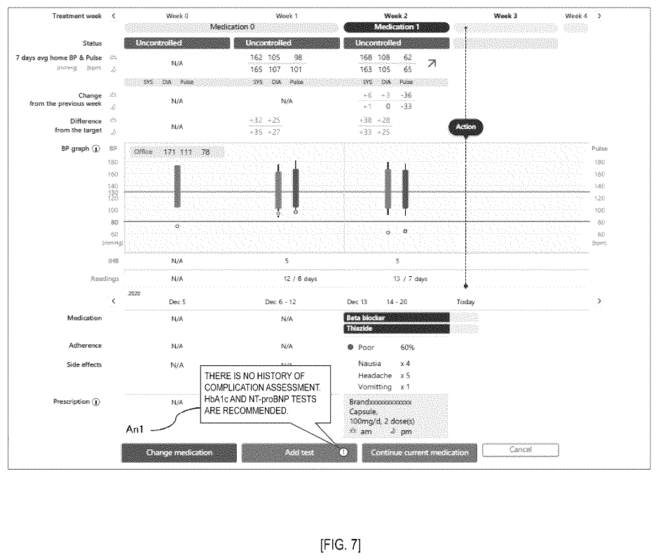

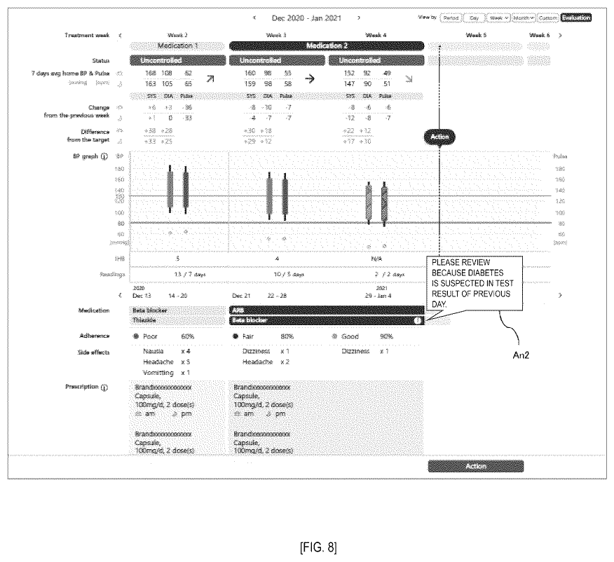

FIG. 7 and FIG. 8 are each an example of a screen in which recommended treatment information An1 and An2 are displayed in a pop-up manner on the treatment status confirmation image. In the screen example of FIG. 7, since information for selecting an appropriate guideline is insufficient, the recommended treatment information An1 for recommending additional tests to be performed on the patient is displayed. In the screen example of FIG. 8, since an appropriate guideline (recommended prescription pattern) is different from the prescription content currently applied to the patient, the recommended treatment information An2 for recommending a prescription content matching the recommended prescription pattern is displayed (the prescription pattern of diabetes complication is recommended).

By referring to such recommended treatment information, the doctor can easily determine what kind of action (including observation) should be taken on the patient P. Note that the recommended treatment information need not be output together with the treatment status confirmation image, and may be displayed after transition to another screen, or may be displayed regardless of the treatment status confirmation image.

### Patient-Side Terminal

The patient-side terminal 300 is, for example, a portable information processing terminal, such as a smartphone, a tablet terminal, or a wristwatch type wearable terminal, and although not illustrated, includes a control unit, such as a CPU, input/output means, such as a touch panel display, storage means, and communication means, such as a wireless communication circuit.

In addition to measurement data acquired from the blood pressure monitor 400 described below, the patient-side terminal 300 transmits information related to treatment of a disease, such as a medicine ingestion status and lifestyle habits, that is input as necessary by the patient P to the server device 100.

### Blood Pressure Monitor

The blood pressure monitor 400 is used by the patient P for daily blood pressure measurement, and may have any kind of form. For example, it may be a general stationary type, a portable type in which a portable cuff is wrapped around an upper arm or the like, or may be of a wearable type to be worn on a wrist of a patient. The blood pressure is measured by the oscillometric method by an operation of the patient P (or, in the case of a wearable type, at a timing or time interval set in advance), and the blood pressure data is wirelessly transmitted to the patient-side terminal 300 by wireless communication, for example. As a communication interface used between the blood pressure monitor 400 and the patient-side terminal 300, a short-range wireless data communication standard, such as Bluetooth (trade name) or infrared communication, can be adopted.

The blood pressure monitor 400 need not have communication means. In that case, the patient P may manually input measurement data (including a blood pressure value and a measurement date and time) to the patient-side terminal 300, and the information may be transmitted to the server device 100. Note that the measurement data may include heart rate.

Further, the patient-side terminal 300 may also have the function of the blood pressure monitor 400. For example, in a case where the patient-side terminal 300 is a wearable terminal worn on the wrist of the patient P, when a blood pressure measurement function is provided in the wearable terminal, the wearable terminal can also serve as the blood pressure monitor 400. Conversely, for example, the stationary blood pressure monitor 400 may have a function as an information processing terminal, and also serve as the patient-side terminal 300.

The measurement method of the blood pressure monitor 400 may be a method in which measurement is performed for each heartbeat, or a trigger measurement method in which blood pressure fluctuation is estimated from pulse transit time and blood pressure is measured in a spot manner using the fluctuation as a trigger.

### Flow of Recommended Treatment Information Output Processing

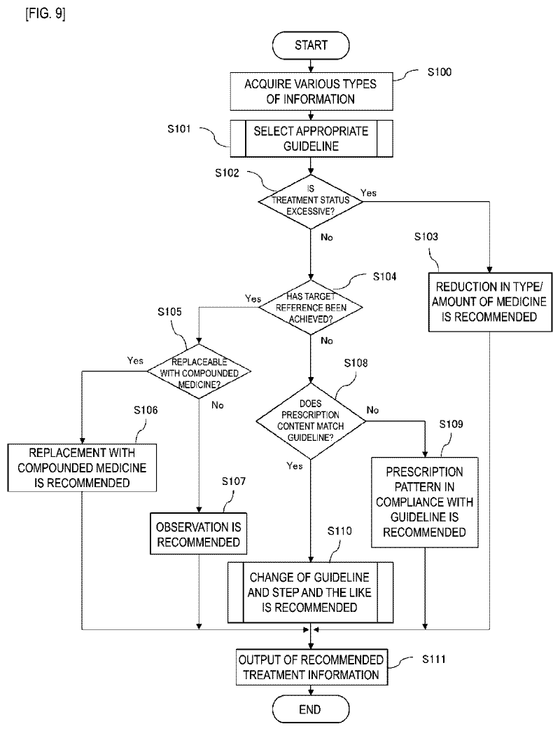

Next, a flow of recommended treatment information output processing performed by the medical care assistance system 1 according to the present example having the above-described configuration will be described with reference to FIG. 9. FIG. 9 is a flowchart illustrating a flow of processing for guideline selection, appropriateness determination, and creation and output of recommended medical information performed in the medical care assistance system 1.

Although not illustrated, the recommended treatment information output processing in the present example starts by being triggered by a request from the doctor-side terminal 200, arrival of a predetermined confirmation time, or the like. First, the server device 100 acquires various kinds of information including patient information (S100). Note that the acquisition of information here includes reading out various kinds of information acquired via the communication network N and stored in the storage means 120 prior to execution of the processing. Then, the guideline selection unit 112 of the server device 100 selects a guideline/prescription pattern related to the treatment of the patient P (S101). The details of the selection processing have already been described in the description of the guideline selection unit 112, and thus will not be described again.

Next, the appropriateness determination unit 113 determines whether or not the current prescription content is excessive for the medical condition of the patient P using the patient information (measured value of blood pressure), a blood pressure lower limit value set in advance, and the like as input information (S102). Here, in a case where it is determined that the current prescription content is excessive for the medical condition of the patient P (for example, the measured value is lower than the lower limit value), the recommended treatment information generation unit 115 generates recommended treatment information that recommends reduction in type/amount of medicine, (S103), and the flow proceeds to step S111.

On the other hand, when it is determined in step S102 that the current prescription content is not excessive for the medical condition of the patient P, the appropriateness determination unit 113 subsequently determines whether or not the treatment target has been achieved by using the patient information, the predetermined improvement target reference value, and the like as input information (S104). Here, when it is determined that the treatment target has been achieved (the measured value is lower than the improvement target reference value), the flow proceeds to step S105, and it is determined whether or not any of the currently prescribed medicines is replaceable with a compounded medicine (S105). Specifically, for example, the determination can be made by comparing the compounded medicine list stored in the medicine information storage unit 124 with the prescription content. When it is determined in step S105 that there is a current prescribed medicine that is replaceable with a compounded medicine, the recommended treatment information generation unit 115 generates recommended treatment information that recommends changing the prescription content to a content in which the currently prescribed medicine has been replaced with the compounded medicine (S106), and the flow proceeds to step S111. On the other hand, when it is determined in step S105 that there is no currently prescribed medicine that is replaceable with a compounded medicine, the recommended treatment information generation unit 115 generates recommended treatment information that recommends observation (S107), and the flow proceeds to step S111.

In step S104, when it is determined that the treatment target has not been achieved, the flow proceeds to step S108, and the appropriateness determination unit 113 determines whether or not the current prescription content matches the recommended prescription pattern of the guideline (S108). Here, when it is determined that the prescription content does not match the recommended prescription pattern of the guideline, the recommended treatment information generation unit 115 generates recommended treatment information that recommends changing to a prescription content in compliance with the recommended prescription pattern (S109), and the flow proceeds to step S111.

On the other hand, when it is determined in step S108 that the current prescription content matches the recommended prescription pattern of the guideline, the recommended treatment information generation unit 115 generates recommended treatment information regarding an action to be performed using patient information, guideline information, disease category information, medicine information, and the like as input information (S110). Specifically, for example, the current step in the guideline is checked based on the current prescription content. Then, in a case where the next step is set in the guideline, the recommended treatment information for changing to the prescription content matching the recommended prescription pattern of the next step (advancing the step of the guideline) is generated. Note that the non-recommended medicine determination unit 114 may determine whether or not the recommended prescription pattern in the next step includes a contraindication or an unacceptable medicine. When a contraindication/unacceptable medicine is included, recommended treatment information that recommends a prescription content based on the contraindication or unacceptable medicine is generated.

In addition, when the next step is not set in the guideline, the recommended treatment information that recommends the performance of additional tests (suggesting changing the guideline) may be generated in consideration of the possibility that the correct guideline is not applied.

Then, the generated recommended treatment information is transmitted to the doctor-side terminal 200, the recommended treatment information is outputted by the output means 230 of the doctor-side terminal (S111), and a series of routines is ended. In the present example, step S100 corresponds to a patient information acquiring step, and step S101 corresponds to a guideline selecting step. In addition, steps S102, S104, S105, and S108 correspond to determination steps, and steps S103, S105, S107, S109, and S110 correspond to recommended treatment information generation steps.

With the medical care assistance system 1 according to the present example as described above, it is possible to provide the recommended treatment information regarding the action recommended to be performed on the patient based on the patient information, the guideline information, and the like. By referring to such recommended treatment information, the doctor can easily determine what kind of treatment (action) should be performed on the patient.

In addition, since the treatment status can be reevaluated based on the measured value of the biological information, the medication information, and the like, it is possible to recommend a change of the prescription content including a change of the guideline to be applied. This makes it possible to recommend an appropriate treatment content to a doctor, for example, even for a patient who has been transferred from another hospital or a patient whose medical condition has changed. In addition, by presenting the recommended treatment information, it is possible to prevent a situation (so-called clinical inertia) in which appropriate treatment reinforcement is not performed even though the treatment target has not been achieved.

### Modified Example

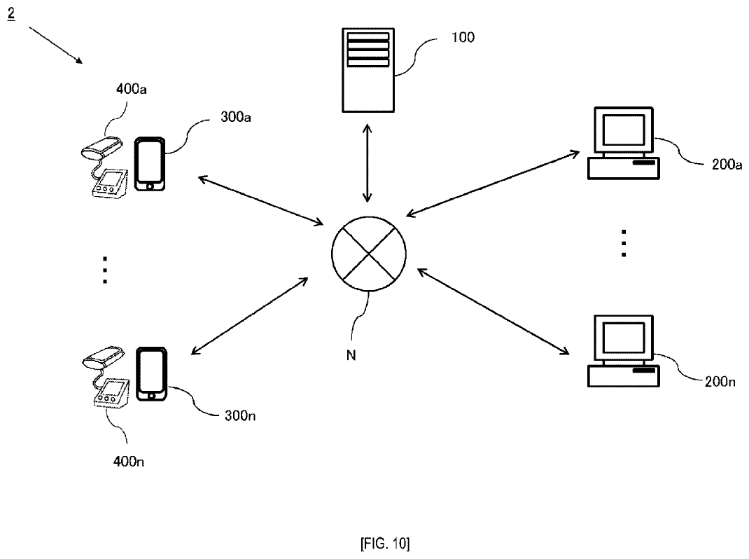

The description of the example described above is merely illustrative of the present invention, and the present invention is not limited to the specific embodiments described above. Within the scope of the technical idea of the present invention, various modifications and combinations may be made. For example, in the above-described example, a configuration including one of each the doctor-side terminal 200 and the patient-side terminal 300 has been described. However, as illustrated in FIG. 10, the present invention can be applied to a medical care assistance system 2 including a plurality of doctor-side terminals 200a to 200n and/or a plurality of patient-side terminals 300a to 300n.

In addition, in the above-described example, the processing of selecting a guideline in step S101 can be appropriately omitted in the second and subsequent medical treatments. Moreover, the above-described flow of processing can be appropriately exchanged. For example, the determination (S102) of whether or not the treatment status is excessive may be performed after it is determined that the treatment target has been achieved and before it is determined whether or not replacement with a compounded medicine is possible (between step S104 and step S105).

Further, in the above-described example, as an example of remote treatment of a hypertensive patient, a case where the biological information to be processed is blood pressure value (and heart rate) has been described. However, it is needless to say that the present invention can be applied to other diseases and biological information, such as blood glucose level of a diabetic patient. Further, the present invention need not be used in combination with remote medical care, and can be applied as an off-line system or device as long as necessary input information can be obtained.

### REFERENCE SIGNS LIST

1, 2 Medical care assistance system
100 Server device
110, 210 Control unit
120, 240 Storage means
130, 250 Communication means
200 Doctor-side terminal
220 Input means
230 Output means
300 Patient-side terminal
400 Blood pressure monitor
P Patient
N Communication network
Tr Measured value transition information
Me Medicine ingestion information
An1, An2 Recommended treatment information

1. A medical care assistance system, comprising:
guideline selection unit that selects a guideline related to treatment of a patient based on predetermined input information;
patient information acquisition unit that acquires patient information including measured value information related to biological information on the patient and medication information as information of a medicine prescribed to the patient;
determination unit that performs determination related to appropriateness of a current treatment strategy including a prescription content of a medicine for the patient based on the selected guideline and the patient information;
recommended treatment information generation unit that generates recommended treatment information regarding an action recommended to be performed on the patient according to a determination result of the determination unit; and
at least one output device that outputs the recommended treatment information, wherein
the determination unit determines at least whether or not the prescription content currently adopted is excessive for a medical condition of the patient based on the patient information; and
the recommended treatment information generation unit generates the recommended treatment information that recommends reduction in type and/or amount of medicine prescribed to the patient when the prescription content currently adopted is excessive for the medical condition of the patient.

2. The medical care assistance system according to claim 1, wherein
the predetermined input information is information including at least any of a medical history, a biochemical test result, an electrocardiographic finding, a left ventricular ejection fraction by echocardiogram, and an arteriosclerosis index; and
the guideline selection unit specifies a disease of the patient based on the predetermined input information, and selects a guideline related to treatment of the disease of the patient.

3. The medical care assistance system according to claim 1, wherein
the determination unit determines at least whether or not the medicine according to the prescription content currently adopted is replaceable with a compounded medicine; and
the recommended treatment information generation unit generates the recommended treatment information that recommends a prescription change to the compounded medicine when the medicine according to the prescription content currently adopted is replaceable with the compounded medicine.

4. The medical care assistance system according to claim 1, wherein
the guideline defines a recommended prescription content according to a medical condition;
the determination unit determines at least whether or not the prescription content currently adopted matches the recommended prescription content; and
the recommended treatment information generation unit generates the recommended treatment information that recommends a prescription change to the recommended prescription content when the prescription content currently adopted does not match the recommended prescription content.

5. The medical care assistance system according to claim 1, wherein
the guideline defines a recommended prescription content according to a medical condition,
the patient information includes a predetermined improvement target reference related to the biological information,
the determination unit determines at least whether the prescription content currently adopted matches the recommended prescription content, and whether the measured value information has achieved the improvement target reference, and
the recommended treatment information generation unit generates, when the measured value information has not achieved the improvement target reference and the prescription content currently adopted matches the recommended prescription content, the recommended treatment information that recommends a prescription change based on the recommended prescription content one step ahead in the guideline or the recommended treatment information that recommends a test to be performed on the patient.

6. The medical care assistance system according to claim 5, further comprising
non-recommended medicine determination unit that determines presence or absence of a non-recommended medicine that is unrecommended to be prescribed to the patient based on the patient information; wherein
the recommended treatment information generation unit generates, when the non-recommended medicine is present, the recommended treatment information that recommends a prescription change to a prescription content excluding the non-recommended medicine.

7. The medical care assistance system according to claim 1, wherein
the predetermined input information includes an international disease classification code related to a disease of the patient.

8. The medical care assistance system according to claim 1, wherein
the biological information includes at least a blood pressure value, and a disease targeted for treatment of the patient is hypertension.

9. A medical care assistance device, comprising:
the guideline selection unit:
the patient information acquisition unit:
the determination unit: and
the recommended treatment information generation unit, wherein
the medical care assistance device constitutes at least a part of the medical care assistance system according to claim 1.

10. A medical care assistance method, comprising:
selecting a guideline related to treatment of a patient based on predetermined input information;
acquiring patient information including measured value information related to biological information of the patient and medication information as information of a medicine prescribed to the patient;
performing determination related to appropriateness of a current treatment strategy including a prescription content of a medicine for the patient based on the selected guideline and the patient information;
generating recommended treatment information regarding an action recommended to be performed on the patient according to a determination result of the performing; and
outputting the recommended treatment information, wherein
the performing determines at least whether or not the prescription content currently adopted is excessive for a medical condition of the patient based on the patient information; and
the generating generates the recommended treatment information that recommends reduction in type and/or amount of medicine prescribed to the patient when the prescription content currently adopted is excessive for the medical condition of the patient.

11. A recording medium for causing a computer to function as the medical care assistance device according to claim 9.

12. The medical care assistance system according to claim 2, wherein
the determination unit determines at least whether or not the medicine according to the prescription content currently adopted is replaceable with a compounded medicine; and
the recommended treatment information generation unit generates the recommended treatment information that recommends a prescription change to the compounded medicine when the medicine according to the prescription content currently adopted is replaceable with the compounded medicine.

13. The medical care assistance system according to claim 3, wherein
the guideline defines a recommended prescription content according to a medical condition;
the determination unit determines at least whether or not the prescription content currently adopted matches the recommended prescription content; and
the recommended treatment information generation unit generates the recommended treatment information that recommends a prescription change to the recommended prescription content when the prescription content currently adopted does not match the recommended prescription content.

14. The medical care assistance system according to claim 2, wherein
the guideline defines a recommended prescription content according to a medical condition,
the patient information includes a predetermined improvement target reference related to the biological information,
the determination unit determines at least whether the prescription content currently adopted matches the recommended prescription content, and whether the measured value information has achieved the improvement target reference, and
the recommended treatment information generation unit generates, when the measured value information has not achieved the improvement target reference and the prescription content currently adopted matches the recommended prescription content, the recommended treatment information that recommends a prescription change based on the recommended prescription content one step ahead in the guideline or the recommended treatment information that recommends a test to be performed on the patient.

15. The medical care assistance system according to claim 3, wherein
the guideline defines a recommended prescription content according to a medical condition,
the patient information includes a predetermined improvement target reference related to the biological information,
the determination unit determines at least whether the prescription content currently adopted matches the recommended prescription content, and whether the measured value information has achieved the improvement target reference, and
the recommended treatment information generation unit generates, when the measured value information has not achieved the improvement target reference and the prescription content currently adopted matches the recommended prescription content, the recommended treatment information that recommends a prescription change based on the recommended prescription content one step ahead in the guideline or the recommended treatment information that recommends a test to be performed on the patient.

16. The medical care assistance system according to claim 4, wherein
the guideline defines a recommended prescription content according to a medical condition,
the patient information includes a predetermined improvement target reference related to the biological information,
the determination unit determines at least whether the prescription content currently adopted matches the recommended prescription content, and whether the measured value information has achieved the improvement target reference, and
the recommended treatment information generation unit generates, when the measured value information has not achieved the improvement target reference and the prescription content currently adopted matches the recommended prescription content, the recommended treatment information that recommends a prescription change based on the recommended prescription content one step ahead in the guideline or the recommended treatment information that recommends a test to be performed on the patient.

17. The medical care assistance system according to claim 2, wherein
the predetermined input information includes an international disease classification code related to a disease of the patient.

18. The medical care assistance system according to claim 3, wherein
the predetermined input information includes an international disease classification code related to a disease of the patient.

19. The medical care assistance system according to claim 4, wherein
the predetermined input information includes an international disease classification code related to a disease of the patient.

20. The medical care assistance system according to claim 5, wherein
the predetermined input information includes an international disease classification code related to a disease of the patient.

21. The medical care assistance system according to claim 6, wherein
the predetermined input information includes an international disease classification code related to a disease of the patient.

22. The medical care assistance system according to claim 2, wherein
the biological information includes at least a blood pressure value, and a disease targeted for treatment of the patient is hypertension.

23. The medical care assistance system according to claim 3, wherein
the biological information includes at least a blood pressure value, and a disease targeted for treatment of the patient is hypertension.

24. The medical care assistance system according to claim 4, wherein
the biological information includes at least a blood pressure value, and a disease targeted for treatment of the patient is hypertension.

25. The medical care assistance system according to claim 5, wherein
the biological information includes at least a blood pressure value, and a disease targeted for treatment of the patient is hypertension.

26. The medical care assistance system according to claim 6, wherein
the biological information includes at least a blood pressure value, and a disease targeted for treatment of the patient is hypertension.

27. The medical care assistance system according to claim 7, wherein
the biological information includes at least a blood pressure value, and a disease targeted for treatment of the patient is hypertension.

28. A medical care assistance device, comprising:
the guideline selection unit:
the patient information acquisition unit:
the determination unit: and
the recommended treatment information generation unit, wherein
the medical care assistance device constitutes at least a part of the medical care assistance system according to claim 2.

29. A medical care assistance device, comprising:
the guideline selection unit:
the patient information acquisition unit:
the determination unit: and
the recommended treatment information generation unit, wherein
the medical care assistance device constitutes at least a part of the medical care assistance system according to claim 3.

30. A medical care assistance device, comprising:
the guideline selection unit:
the patient information acquisition unit:
the determination unit: and
the recommended treatment information generation unit, wherein
the medical care assistance device constitutes at least a part of the medical care assistance system according to claim 4.

31. A medical care assistance device, comprising:
the guideline selection unit:
the patient information acquisition unit:
the determination unit: and
the recommended treatment information generation unit, wherein
the medical care assistance device constitutes at least a part of the medical care assistance system according to claim 5.

32. A medical care assistance device, comprising:
the guideline selection unit:
the patient information acquisition unit:
the determination unit: and
the recommended treatment information generation unit, wherein
the medical care assistance device constitutes at least a part of the medical care assistance system according to claim 6.

33. A medical care assistance device, comprising:
the guideline selection unit:
the patient information acquisition unit:
the determination unit: and
the recommended treatment information generation unit, wherein
the medical care assistance device constitutes at least a part of the medical care assistance system according to claim 7.

34. A medical care assistance device, comprising:
the guideline selection unit:
the patient information acquisition unit:
the determination unit: and
the recommended treatment information generation unit, wherein
the medical care assistance device constitutes at least a part of the medical care assistance system according to claim 8.
